(19) United States
(12) Patent Application Publication
(10) Pub. No.: US 2006/0241360 A1
(43) Pub. Date: Oct. 26, 2006

(54) Living body measuring apparatus

(75) Inventors: James G. Montagnino, St. Charles, IL (US); Toshihiko Ishikawa, Arlington Heights, IL (US); Yuichi Miyashita, Wako-shi (JP)

Correspondence Address: MCDERMOTT WILL & EMERY LLP, 600 13TH STREET, N.W., WASHINGTON, DC, 20005-3096, US

(73) Assignee: TANITA CORPORATION

(21) Appl. No.: 11/184,922

(22) Filed: Jul. 20, 2005

(30) Foreign Application Priority Data
Mar. 4, 2005 (JP) 2005-061123

(51) Int. Cl. A61B 5/00

(52) U.S. Cl. 600/310

(57) ABSTRACT

Disclosed a living body measuring apparatus which comprises data measurement means for measuring plural types of biological data of a user, data selection means for selecting one of the plural types of measured biological data, data display means for displaying the selected biological data, a plurality of light emitters corresponding to the plural types of biological data, and illumination control means for activating one of the light emitters which corresponds to one selected from the biological data by the data selection means. The living body measuring apparatus of the present invention can eliminate the possibility of user's confusion about biological data display and operational complication, at the lowest cost possible.

### BACKGROUND OF THE INVENTION

1. Field of the Invention

The present invention relates to a living body measuring apparatus for measuring a biological data of a user, and more particularly to a living body measuring apparatus including data measurement means for measuring plural types of biological data of a user, data selection means for selecting one of the plural types of measured biological data, and data display means for displaying the selected biological data.

2. Description of the Related Art

There has been widely known an apparatus for measuring plural types of biological data of a user, such as body weight and body fat percentage (see, for example, Japanese Patent Publication No. 05-049050). Particularly, in recent living body measuring apparatuses, the number of kinds or types of measurable biological data has been constantly increased to allow users to measure various biological data, such as visceral fat area, body water percentage, muscle mass, bone mass and basal metabolic rate, as well as body weight and body fat percentage.

In conjunction with increase in the number of types of measurable biological data, data display means equipped in a living body measuring apparatus, such as a liquid-crystal display screen, is liable to have difficulty in displaying an entire measurement result thereon at a time. Thus, one type of living body measuring apparatus has been developed that is designed to automatically select and display each measured biological data, in turn or sequentially and at given time intervals or periodically. In particular, some living body measuring apparatuses of this type are provided with a plurality of key switches corresponding, respectively, to the measurement items, and designed to allow a user to selectively press any one of the key switches so as to display one intended biological data associated with the pressed key switch (see, for example, Japanese Patent Application No. 2004-048406). This technique is intended to allow a user to directly select one biological data to be displayed, from plural types of biological data.

### BRIEF SUMMARY OF THE INVENTION

Among the conventional living body measuring apparatus capable of measuring plural types of biological data, the type adapted to automatically select (switch) and display a measurement result in a sequential manner is likely to have a problem about difficulty in making out each item of successively displayed biological data, as the display is more frequently switched along with increase in the number of biological data types. Specifically, most of the living body measuring apparatuses are designed to display on the liquid-crystal display screen only the value of a measured biological data or measurement result and the unit thereof. Thus, if plural types of biological data having the same unit, such as body weight (kg), body fat mass (kg), muscle mass (kg) and bone mass (kg), are successively displayed, a user is liable to fall into confusion about “which of the biological data a currently displayed value indicates”. While it is readily conceivable to tentatively eliminate this confusion by displaying a character or mark representing each biological data together with the biological data on the liquid-crystal display screen, as in some commercial living body measuring apparatuses employing such data display, the liquid-crystal display screen has only a limited allocatable space for such characters or marks, and this solution practically involves a problem about increase in size of the liquid-crystal display screen itself, which is likely to result in an increased total cost of a living body measuring apparatus.

The living body measuring apparatus of the type designed to arbitrarily display a measurement result according to a user's selection operation allows a user to switch and select a biological data to be displayed, on his/her own free will, and thereby the possibility of the above confusion can be reduced. In reality, if the living body measuring apparatus has only one or two key switches for selecting (switching) the display item, a user is required to perform a selection operation while taking account of “how many times the key switch has to be pressed to reach the intended biological data” or “how many times the key switch has been pressed until now”, and the operation itself inevitably becomes complicated. While an apparatus with a plurality of key switches corresponding, respectively, to all of biological data types, allows for selecting one biological data to be displayed, directly by a user, a user is required to select one specific key switch corresponding to one intended biological data, from the key switches arranged on a casing of the apparatus in the same number as that of the items. When the number of biological data types is less than three or four, this operation would not cause any problem. However, if the number is gradually increased to five or six, and further to seven, eight, nine - - - , the key-switch selection itself will become significantly complicated, which is liable to cause user's confusion.

In view of the above problems in the conventional living body measuring apparatus including data measurement means for measuring plural types of biological data of a user, data selection means for selecting one of the plural types of measured biological data, and data display means for displaying the selected biological data, it is therefore an object of the present invention to provide a living body measuring apparatus free from the possibility of the user's confusion about biological data display and the operational complication as described above, at the lowest cost possible.

In order to achieve this object, the present invention provides a living body measuring apparatus comprising data measurement means for measuring plural types of biological data of a user, data selection means for selecting one of the plural types of measured biological data, data display means for displaying the selected biological data, a plurality of light emitters corresponding to the plural types of biological data, and illumination control means for activating one of the light emitters which corresponds to one selected from the biological data by the data selection means.

In the living body measuring apparatus of the present invention, each of the light emitters may include a light-transparent film covering thereover and having a character or mark distinctively representing each biological data associated therewith.

In the living body measuring apparatus of the present invention, the data selection means may include automatic data-selection means for automatically selecting each of the plural types of biological data in turn at given time intervals, and optional data-selection means for selecting one of the plural types of biological data according to a selection operation performed by the user. In this case, the illumination control means may be operable to change a illumination mode of the light emitters depending on whether the selection of the biological data is performed by the automatic data-selection means or the optional data-selection means.

The living body measuring apparatus of the present invention may further comprise data storage means for storing the plural types of biological data measured by the data measurement means. Further, the data selection means may include latest-data selection means for selecting one of the latest biological data measured by the data measurement means, and past-data selection means for selecting one of past biological data stored on the data storage means. In this case, the illumination control means may be operable to change a illumination mode of the light emitters depending on whether the selection of the biological data is performed by the latest-data selection means or the past-data selection means.

In the living body measuring apparatus of the present invention, the data measurement means may include an input key for inputting at least a body height data of the user, a weight sensor for measuring a body weight data of the user, and a combination of an electrode and an electric circuit, for measuring a data about an impedance between two legs of the user. In this case, the plural types of biological data may consist of a plurality of index values about a body composition of the user calculated based on at least the body height, body weight and impedance data.

According to the living body measuring apparatus of the present invention, when one biological data selected by the data selection means is displayed on the data display means, one light emitter associated with the selected biological data is activated by the illumination control means. This allows the user to readily recognize the type of the currently displayed biological data on the data display means, without any confusion. In addition, the use of these light emitters makes it possible to completely omit or otherwise simplify the display of characters or marks representing the biological data in the data display means, so as to avoid the problem about increase in size of the data display means and the resulting problem about increase in total cost of the living body measuring apparatus.

As one of measures for clarifying the correspondence between the light emitters and the biological data in the living body measuring apparatus of the present invention, a character or mark may be labeled or formed on the vicinity of each of the corresponding light emitters. In particular, each of the light emitters may be covered with the light-transparent film having a character or mark distinctively representing each biological data associated therewith to provide excellent visibility and enhanced entire appearance of the apparatus.

Further, when the data selection means in the living body measuring apparatus of the present invention includes both the automatic data-selection means and the optional data-selection means so as to arbitrarily perform the selection of the biological data to be displayed on the data display means, automatically in turn at given time intervals or according to user's selection operation, the illumination mode of the light emitters is changed depending on the respective cases. This makes it possible for the user to readily recognize the difference in current display state, such as whether the display switching is performed in an automatic manner or in a manual manner requiring a user's operation, so as to provide enhanced user-friendliness.

When the living body measuring apparatus of the present invention further comprises the data storage means for storing the plural types of biological data measured by the data measurement means, and the data selection means includes both the latest-data selection means and the past-data selection means so as to select the biological data to be displayed on the data display means, from the latest biological data measured by the data measurement means or past biological data stored on the data storage means, the illumination mode of the light emitters is changed depending on the respective cases. This makes it possible for the user to readily recognize the difference in current display state, such as whether the data display means displays a newly measured biological data or a previously measured biological data, so as to provide enhanced user-friendliness.

The data measurement means in the living body measuring apparatus of the present invention may be composed of an input key for inputting at least a body height data of the user, a weight sensor for measuring a body weight data of the user and a combination of an electrode and an electric circuit for measuring a data about an impedance between two legs of the user, so as to measure the plural types of measured biological data which can serve as a plurality of index values about a body composition of the user calculated based on at least the body height, body weight and impedance data. In this way, the living body measuring apparatus of the present invention can be achieved as an apparatus for measuring a body composition of a user.

### BRIEF DESCRIPTION OF THE DRAWINGS

FIG. 1 is a perspective external view of a living body measuring apparatus 1 according to one embodiment of the present invention.

FIG. 2 is a schematic block diagram of an electric circuit configuration incorporated in the living body measuring apparatus 1.

FIG. 3 is a flowchart of a personal-data registration process.

FIG. 4 is a flowchart of a biological-data measurement process.

FIG. 5 is a flowchart of a latest-biological-data display process.

FIG. 6 is a flowchart of a past-biological-data display process.

FIGS. 7(A) to 7(D) are explanatory diagrams of various examples of a display of a liquid-crystal display screen and a illumination mode of light emitters.

### DETAILED DESCRIPTION OF THE INVENTION

A living body measuring apparatus of the present invention comprises data measurement means for measuring plural types of biological data of a user, data selection means for selecting one of the plural types of measured biological data, data display means for displaying the selected biological data, a plurality of light emitters corresponding to the plural types of biological data, and illumination control means for activating one of the light emitters which corresponds to one selected from the biological data by the data selection means.

Preferably, each of the light emitters is covered by a transparent cover having characters or a sign representing biological data corresponding to said light emitter.

In the living body measuring apparatus of the present invention, the data selection means preferably includes automatic data-selection means for automatically selecting each of the plural types of biological data in turn at given time intervals, and optional data-selection means for selecting one of the plural types of biological data according to a selection operation performed by the user, wherein the illumination control means is operable to change a illumination mode of the light emitters depending on whether the selection of the biological data is performed by the automatic data-selection means or the optional data-selection means.

Preferably, the living body measuring apparatus of the present invention further comprises data storage means for storing the plural types of biological data measured by the data measurement means, wherein the data selection means includes latest-data selection means for selecting one of the latest biological data measured by the data measurement means, and past-data selection means for selecting one of past biological data stored on the data storage means, and wherein the illumination control means is operable to change a illumination mode of the light emitters depending on whether the selection of the biological data is performed by the latest-data selection means or the past-data selection means.

In the living body measuring apparatus of the present invention, the data measurement means preferably includes an input key for inputting at least a body height data of the user, a weight sensor for measuring a body weight data of the user, and a combination of an electrode and an electric circuit, for measuring a data about an impedance between two legs of the user, wherein the plural types of biological data are a plurality of index values about a body composition of the user calculated based on at least the body height, body weight and impedance data.

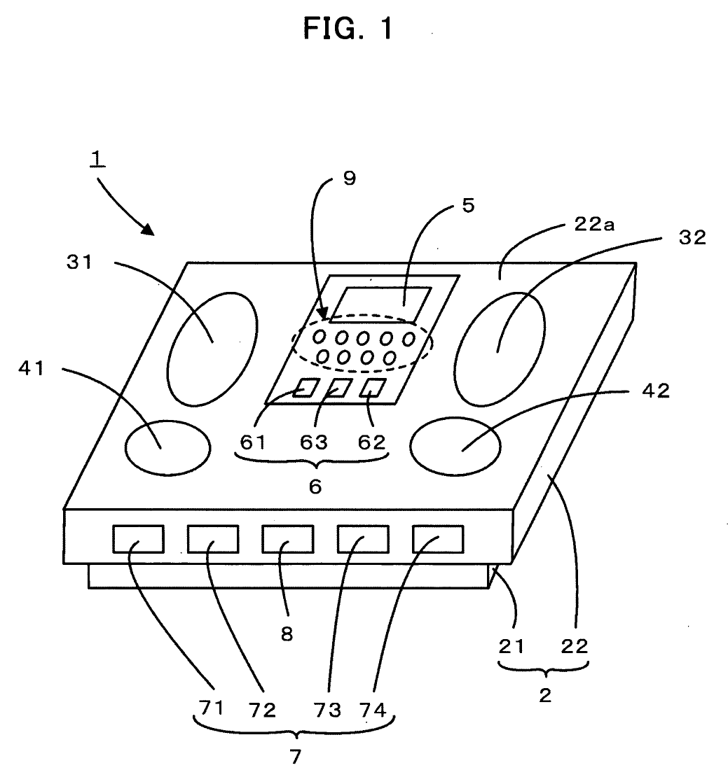

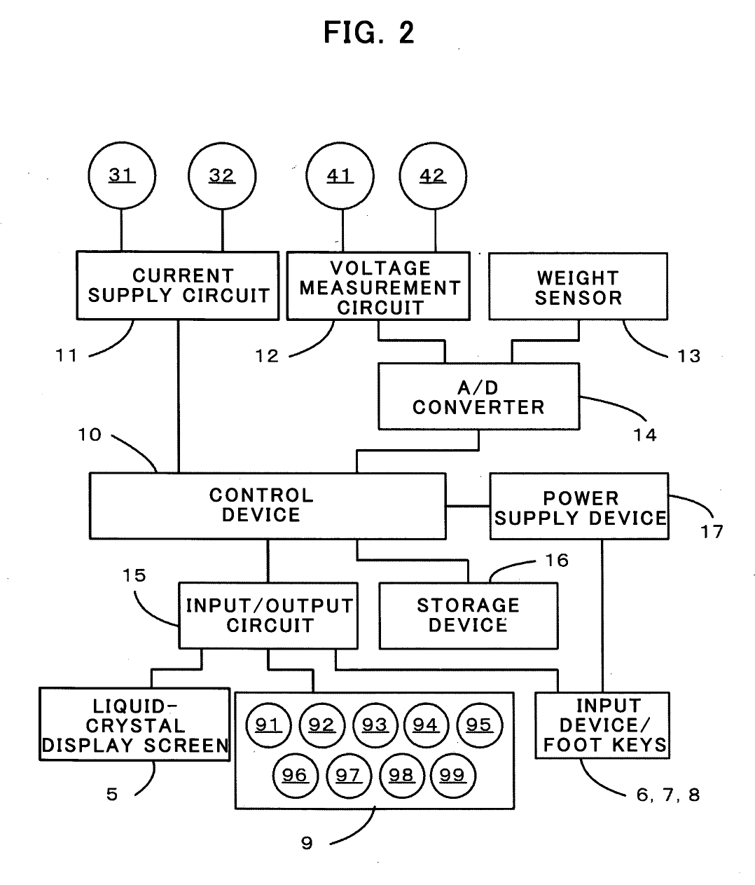

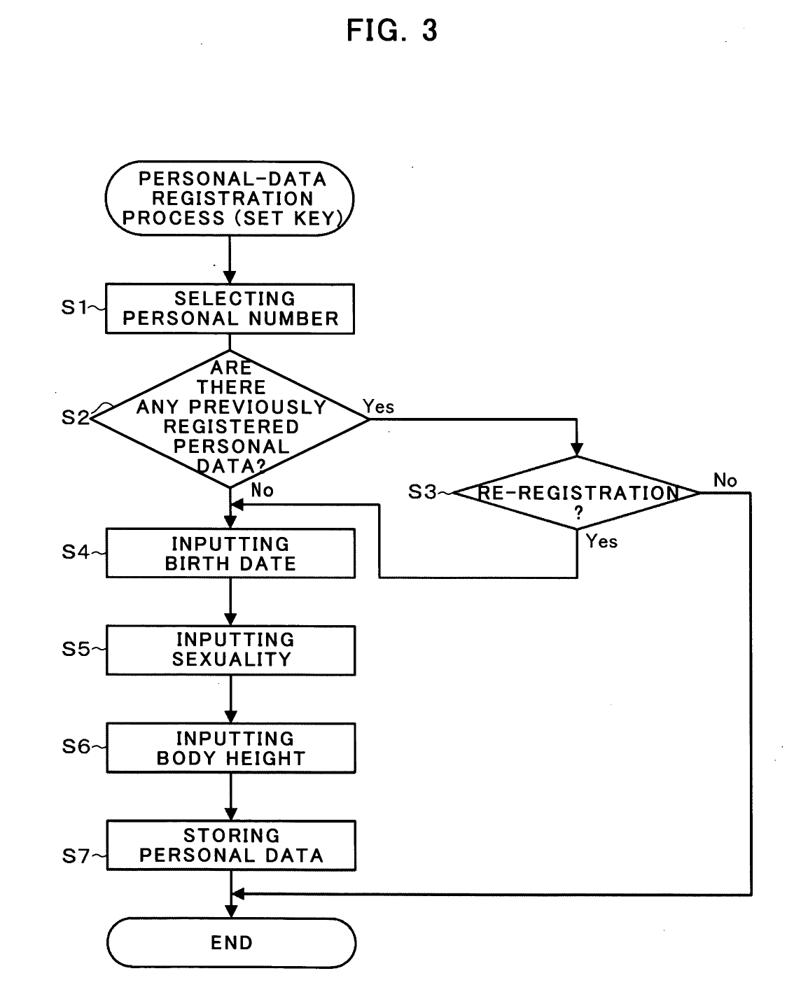

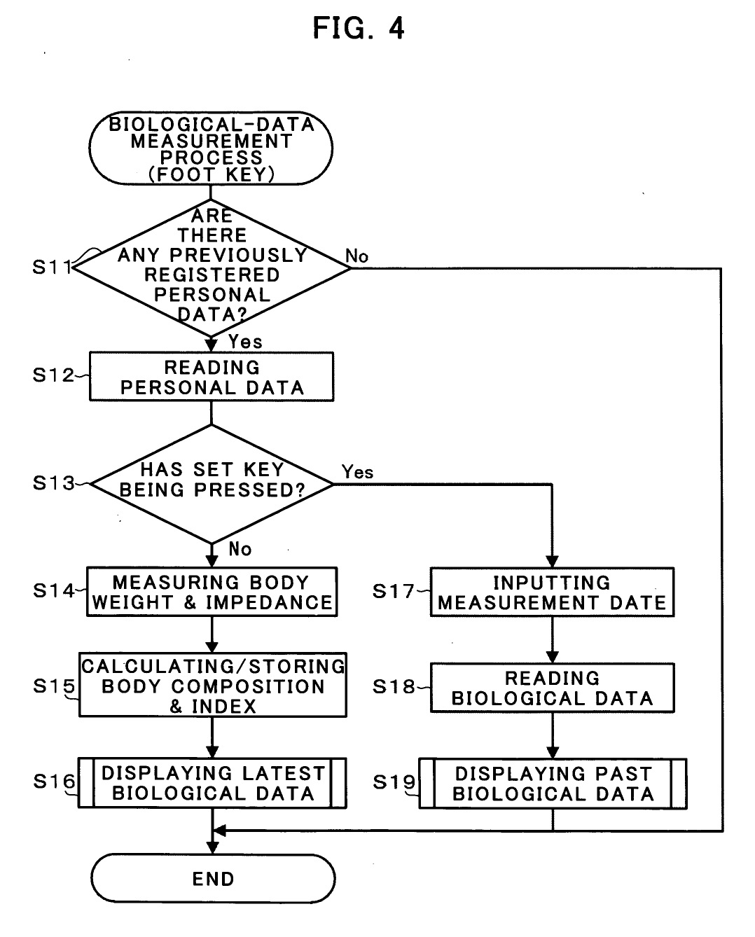

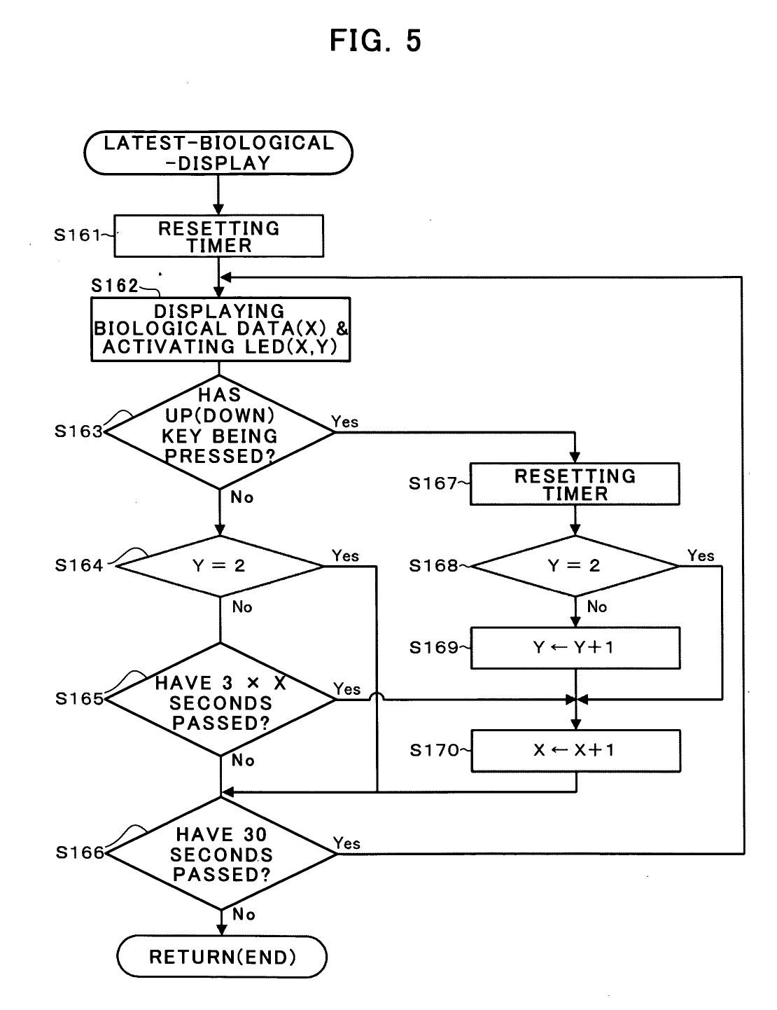

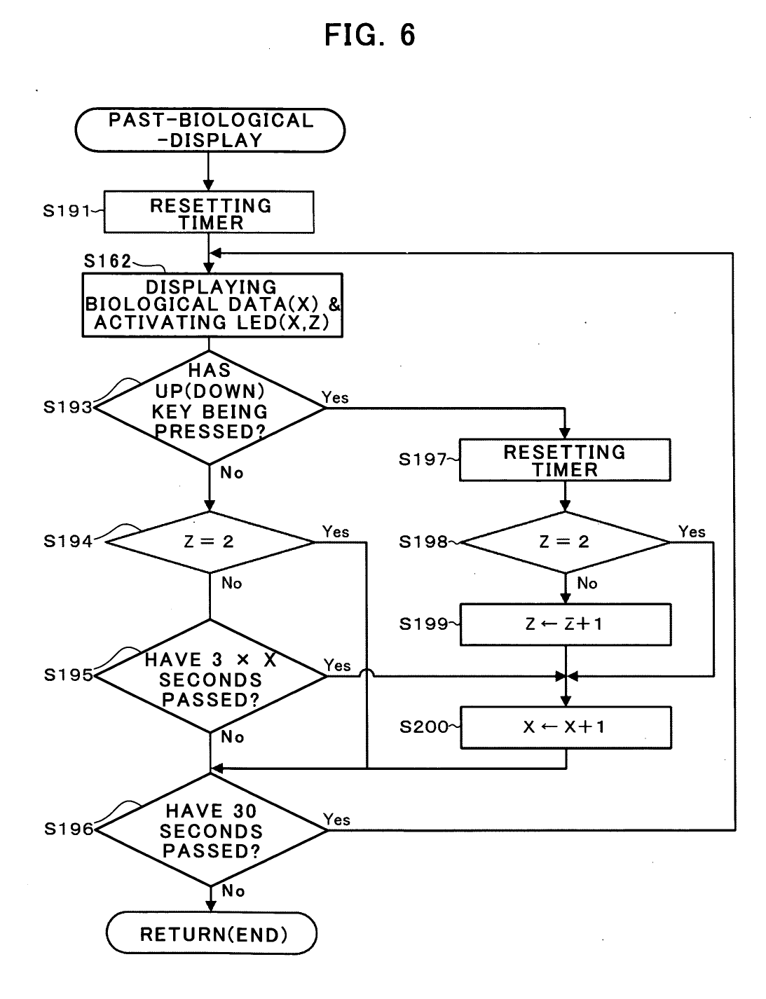

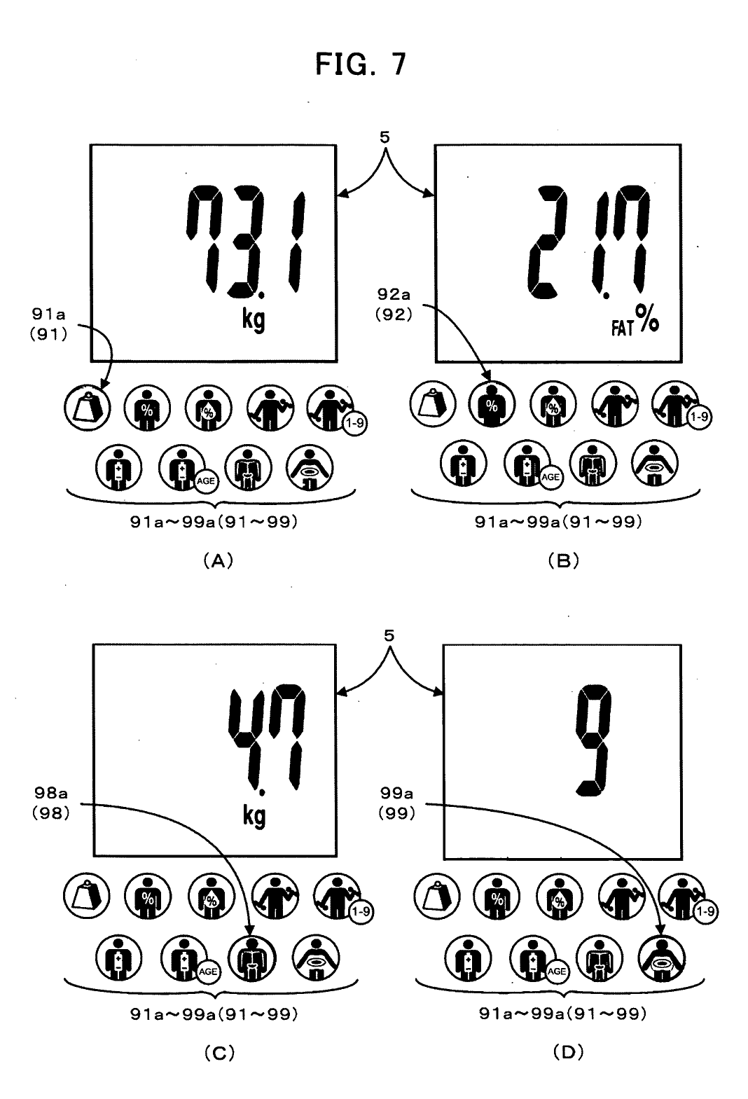

A preferred embodiment of the present invention will now be described with reference to the drawings, wherein: FIG. 1 is a perspective external view of a living body measuring apparatus 1 according to one embodiment of the present invention; FIG. 2 is a schematic block diagram of an electric circuit configuration incorporated in the living body measuring apparatus 1; FIG. 3 is a flowchart of a personal-data registration process to be executed in the living body measuring apparatus 1; FIG. 4 is a flowchart of a biological-data measurement process to be executed in the living body measuring apparatus 1; FIG. 5 is a flowchart of a latest-biological-data display process to be executed in the living body measuring apparatus 1; FIG. 6 is a flowchart of a past-biological-data display process to be executed in the living body measuring apparatus 1; and FIGS. 7(A) to 7(D) are explanatory diagrams of various examples of a display of a liquid-crystal display screen and a illumination mode of light emitters, in the living body measuring apparatus 1.

This living body measuring apparatus 1 (hereinafter referred to as “apparatus 1” for brevity) is an improved type of the so-called “body composition analyzer” designed to calculate body composition data of a user, such as body fat percentage, body water percentage, muscle mass, basal metabolic rate or bone mass, by use of user's data about age, sexuality, body height, body weight and inter-foot impedance (bioelectric impedance between feet). Specifically, based on the above user's data, the apparatus 1 is adapted to additionally calculate a biological index data, such as visceral fat level, physique level or metabolic age. In the following description, the above age, sexuality and body height data will hereinafter be referred to collectively as “personal data”, and the total nine data consisting of the body weight data, the five body composition data and the three biological index data will hereinafter be referred to collectively as “biological data”.

As shown in FIG. 1, the apparatus 1 has a main body 2 comprising a base 21 and a load-receiving platform 22 mounted on the base 21. The platform 22 has a top surface 22a provided with a pair of current supply electrodes 31, 32 for supplying an AC current between the right and left feet bottoms of a user, a pair of measurement electrodes 41, 42 for measuring a voltage (potential difference arising between the right and left feet in response to the current supply, a liquid-crystal display screen 5 for displaying personal data and biological data, and an input device 6 for allowing the user to input personal data therethrough.

The input device 6 includes an up key 61, a down key 62 and a set key 63 which are used in an operation for selecting and setting a data or data to be displayed on the liquid-crystal display screen 5. The main body 2 has a side surface a set of foot keys 7 consisting of four foot keys 71, 72, 73, 74 which are used for calling up registered personal data if any, and a foot key 8 for turning off power. Each of the set key 63 and the foot keys 71 to 74 doubles as a power-on key. Further, as indicated by the dashed circle in FIG. 1, the top surface 22a of the platform 22 is provided with a set of light emitter 9 consisting of after-mentioned nine LEDs (light-emitting diodes) 91, 92, 93, 94, 95, 96, 97, 98, 99.

As shown in FIG. 2, the apparatus 1 is internally provided with a current supply circuit 11 electrically connected to the current supply electrodes 31, 32, a voltage measurement circuit 12 electrically connected to the measurement electrodes 41, 42, a weight sensor 13 adapted to generate an output corresponding to a load applied thereon so as to measure body weight data of the user, an A/D converter 14 for converting a voltage signal from the voltage measurement circuit 12 and the weight sensor 13 to a digital signal, an input/output circuit 15 electrically connected to each of the liquid-crystal display screen 5, the input device 6, the foot keys 7, the power-off key 8 and the light emitters 9 (LEDs 91 to 99), a storage device 16 for storing the input personal data and the measured biological data, a power supply device 17 including a battery, and a control device 10 electrically connected to each of the current supply circuit 11, the A/D converter 14, the input/output circuit 15, the storage device 16 and the power supply device 17.

The above control device 10 is provided with a conventional computing element (CPU), and adapted to continuously update current date/hour according to a built-in clock and execute a control program pre-stored on the storage device 16 so as to measure biological data of a user by performing various control processes, such as a process of receiving input personal data from the input device 6; a process of measuring body weight data using the weight sensor 13; a process of supplying an AC current to the current supply electrodes 31, 32; a process of calculating a bioelectric impedance of the user in accordance with the supplied current value and a voltage value detected by the measurement electrodes 41, 42; a process of calculating body composition data and biological index data in accordance with the calculated bioelectric impedance, the input personal data from the input device 4 and the measured body weight data from the weight sensor 13; a process of displaying the input personal data and the measured biological data on the liquid-crystal display screen 5; and a process of storing these data on the storage device 16.

Particularly, in the apparatus 1 according to this embodiment, the light emitters 9 (LEDs 91 to 99) correspond, respectively, to nine types of biological data to be measured by this apparatus 1, and the control device 10 is operable to perform a control process of displaying each of the biological data on the liquid-crystal display screen 5, in an automatic sequential/periodical manner, or according to a selection operation of the user, and lighting or activating the light emitter 9 corresponding to the displayed biological data. With reference to FIGS. 3 to 7(D), these control processes will be described below.

The flowchart of FIG. 3 shows a process of registering personal data, for example, when a user uses the apparatus 1 for the first time. When a user presses down the set key 63 to activate the apparatus 1, this registration process is performed.

In Step S1, a personal number of the user is determined. Specifically, four numerals 1 to 4 of alternative personal numbers are firstly displayed on the liquid-crystal display screen 5, and the user selects any one of the displayed personal numbers using the up key 61 and/or the down key 62 and sets it as his/her personal number using the set key 63. Then, in Step S2, it is determined whether there are any previously registered personal data corresponding to the selected personal number. When the determination in Step S2 is YES or there are the reregistered personal data, the process advances to Step S3. If the determination in Step S2 is NO or there is no registered personal data, the process will advance to Step S4. In Step S3, a selection message requesting to determine whether a re-registration is performed is displayed on the liquid-crystal display screen 5. If the user selects the re-registration, the process will advance to Step S4. When the user selects the non-re-registration, the registration process is terminated and the power supply is automatically turned off.

In Step S4, the birth date of the user is input. Specifically, numerals representing year, month and day are displayed on the liquid-crystal display screen 5, and the user increases/reduces each of the numerals using the up key 61 and/or the down key 62 to adjust the numerals to his/her own birth date, and sets the adjusted birth date using the set key 63. Then, the control device 10 compares the input birth date with a current date indicated by the built-in clock to calculate an age data. Then, in Step S5, a sexuality data of the user is input. Specifically, a selection message requesting to select either one of man and woman is displayed on the liquid-crystal display screen 5, and the user selects his/her sexuality using the up key 61 and/or the down key 62, and sets the selected sexuality using the set key 63. Then, in Step S6, a body height data of the user is input. Specifically, numerals representing body height are displayed on the liquid-crystal display screen 5, and the user increases/reduces each of the numerals using the up key 61 and/or the down key 62 to adjust the numerals to his/her own body height, and sets the adjusted body height using the set key 63.

In Step S7, the personal number selected in Step S1 and the personal data input in Steps S4 to S6 are associated with each other, and stored on the storage device 16. Through the above steps, the personal-data registration process is completed, and, after the lapse of a given time from the completion, the power supply will be automatically turned off. During this time period, the input personal data may be displayed on the liquid-crystal display screen 5 to allow the user to confirm them. It is to be understood that the user may press the power-off key 8 to turn off power.

The flowchart of FIG. 4 shows a process of measuring biological data, such as user's own body composition or biological index using the apparatus 1. When the user presses down one of the foot keys 7 (71 to 74) which corresponds to the personal number associated with the user's own personal data to activate the apparatus 1, this measurement process is performed.

In Step S11, it is determined whether there are any previously registered personal data corresponding to the pressed foot key 7 selected personal number. If the determination in Step S11 is YES or there are the reregistered personal data, the process will advance to Step S12 to read the registered personal data and then advance to Step S13. When the determination in Step S1 is NO or there is no registered personal data, the measurement process is terminated. Before the termination, a message indicative of non-registration of personal data may be displayed on the liquid-crystal display screen 5 for a given period of time to prompt the user to register his/her personal data, or the process may be shifted to Step S4 in FIG. 3.

In Step S13, it is determined whether the set key 63 has been pressed down. If the determination in Step 13 is YES or the set key 63 has been already pressed down at this moment, the process will advance to Step S17 in which the control device 10 will perform the after-mentioned process of displaying stored past biological data associated with the personal number. When the determination in Step S13 is NO or the weight sensor detects a load without pressing of the set key 63, the process advances to Step S14 to measure biological data.

In Step S14, data about a body weight of the user and a bioelectric impedance between two legs of the user are measured. Specifically, when the user stands on the top surface 22a of the platform 22 in the posture where the left foot bottom is in contact with the current supply electrode 31 and the measurement electrode 41 and the right foot bottom is in contact with the current supply electrode 32 and the measurement electrode 42, the body weight data of the user is measured based on a load detected by the weight sensor 13. Simultaneously, an AC current is supplied between the right/left foot bottoms through the current electrodes 31, 32, and a voltage (potential difference) between the right/left foot bottoms through the measurement electrodes 41, 42. Then, an inter-foot bioelectric impedance is calculated based on the supplied current value and the measured voltage. Alternatively, a plurality of reference resistors each having a different known resistance value may be provided in the electric circuit in series or parallel to the user's body, to obtain a potential differences caused by each of the resistors together with a potential difference arising between the user's feet, and calculate a user's bioelectric impedance in accordance with the ratio between each of the obtained potential differences and each resistance value of the reference resistors. In this case, the impedance data can be obtained even if the current value to be supplied to the user's body is unknown.

Then, in Step S15, body composition data, such as body fat percentage, body water percentage, muscle mass, basal metabolic rate and/or bone mass, and biological index data, such as visceral fat level, physique level and/or metabolic age, are calculated based on the registered age, sexuality and body height data serving as the personal data of the user, and the measured body weight and bioelectric impedance data obtained in Step S14. Then, the calculated biological data or the body composition and biological index data, and the biological data measured in Step S14 or the body weight data, are stored on the storage device 16 in association with the personal number of the user. Each of the above body composition and biological index data are calculated according to individual calculation formulas pre-stored on the storage device 16. Most of the respective calculation formulas are prepared by using all or a part of the above personal data, weight data and impedance data, as parameter. Particularly, in the calculation formulas for body fat percentage and body water percentage as fundamental biological data, at least data about body height body weight and bioelectric impedance are incorporated therein as parameters. The step of measuring biological data using such calculation formulas is the same as that in the conventional body composition analyzer, and it is not related directly to the feature of the present invention. Thus, its further detailed description will be omitted.

Then, in Step S16, each of the latest biological data measured at Steps S14 and S15 is displayed on the liquid-crystal display screen 5 while lighting or activating the corresponding light emitter 9. The control in Step S16 will be described below with reference to the flowchart of FIG. 5.

In Step S161, a timer for controlling a display time-period of each biological data is firstly reset. This timer determines an elapsed time from the initiation of this latest-biological-data display process. The built-in clock of the control device 10 is used as the timer.

Then, in Step S162, the display of each of the biological data onto the liquid-crystal display screen 5 and the lighting or activation of the corresponding light emitter 9 are performed according to a first designation number X programmed in such a manner that it is initially set to “1” and incremented one-by-one in the after-mentioned Step S170 (wherein the value of the first designation number X is returned to “1” after it reaches “9”), and a second designation number Y programmed in such a manner that it is initially set to “1” and incremented by one in the after-mentioned Step S169.

The first designation number X is a parameter for determining the biological data to be displayed and the light emitter 9 to be activated. More specifically, designation numbers “ 1” to “9” are pre-assigned, respectively, to the nine types of biological data to be measure by the apparatus 1 and the corresponding nine LEDs 91 to 99, and the control device 10 is operable, in response to a current value of the first designation number X, to select the biological data assigned with the current value to display it on the liquid-crystal display screen 5, and simultaneously select the light emitter 9 assigned with the current value to activate it.

The second designation number Y is a parameter for determining a illumination mode of the light emitters 9 during the display of the latest biological data. Specifically, the lighting pattern or mode of the light emitters 9 during the display of the latest biological data includes two types: “normal lighting” and “medium-speed blinking”, which are assigned, respectively, with designation numbers “1” and “2”. The control device 10 is operable, in response to a current value of the second designation number Y, to activate the light emitter 9 in the illumination mode assigned with the current value.

Then, in Step S163, it is determined whether the up key 61 or the down key 62 is pressed down. If the determination in Step S163 is NO or none of the up key 61 and the down key 62 is pressed down, the process will advance to Step S164 to display the biological data and activate the corresponding light emitters 9 in accordance with the automatic sequential/periodical data selection. When the determination in Step S163 is YES or either one of the up key 61 and the down key 62 is pressed down, the process advances to Step S167 to display the biological data and activate the corresponding light emitters 9 according to the user's selection operation.

In Step S164, it is determined whether the second designation number Y about the illumination mode has a value of “2”. If the determination in Step S164 is YES or the second designation number Y=“2”, the process will skip Step S165 and advance to Step S166. When the determination in Step S164 is NO or the second designation number Y≠“2” (or Y=“1”), the process advances to Step S165.

The second designation number Y is initially set to “1” and incremented by one in the after-mentioned Step S169 to which the process advances when the determination in Step S163 is YES or either one of the up key 61 and the down key 62 is pressed down. Thus, the second designation number Y is kept at “1” (Y≠“2”) unless the user presses down either one of the up key 61 and the down key 62 once, and thereby the process will advance to Step S165. In contrast, when the user presses down either one of the up key 61 and the down key 62 once, the second designation number Y is changed to “2” in Step S169 as described later. Subsequently, the process will always skip Step S165 and advance to Step S166.

In Step S165, it is determined whether 3×x seconds have passed from the timer reset in Step S161. When the determination in Step S165 is YES or 3×x seconds have passed, the process advances to the after-mentioned Step S170 to increment the first designation number X by one and then advance the process to Step S166. If the determination in Step S165 is NO or 3×x seconds have not passed, the process will advance to Step S166 without going through Step S170. In Step S166, it is determined whether 30 seconds have passed the timer reset in Step S161 or the after-mentioned Step S167. If the determination in Step S166 is YES or 30 seconds have passed, the biological-data display process will be fully completed. When the determination in Step S166 is NO or 30 seconds have not passed, the process returns to Step S162.

Fundamentally, the same value as that of the first designation number X is assigned to the value x of the 3×x seconds in Step S165. For example, when the first designation number X is “1”, the value x is “1”, and it is determined whether 3 (3×1) seconds have passed from the timer reset in Step S161. When the first designation number X is “5”, the value x is “5”, and it is determined whether fifteen (3×5) seconds have passed from the timer reset in Step S161. While the value of the first designation number X is programmed in such a manner as to be returned to “1” after it reaches “9”, the value x is programmed in such a manner as to be stepwise increased to 10, 11, 12, - - - even after it reaches “9”.

Through Steps S161 to S166 and Step S170, the biological-data display and the lighting of the corresponding light emitters 9 are performed based on the automatic sequential/periodical data selection. Specifically, in Step S162, the biological data with the first designation number X of “1” is firstly displayed. Then, when 3 seconds have passed without pressing of the up key 61 and the down key 62, the value of the first designation number X is set to “2”, and the biological data with the first designation number X of “2” is switchingly displayed. Subsequently, the biological data with the first designation number X of “3” is switchingly displayed when 3 seconds have further passed (after 6 (3×2) seconds from the timer reset), and the biological data with the first designation number X of “4” is switchingly displayed when 3 seconds have further passed (after 9 (3×3) seconds from the timer reset). In this manner, each of the nine types of biological data will be automatically selected and displayed on the liquid-crystal display screen 5, in turn at 3-second intervals. In conjunction with this display, the light emitter 9 assigned with the same first designation number X as that of the selected biological data is activated (lighted) in the illumination mode defined by the second designation number Y of “1”. These display and illumination controls will continue for total 30 seconds from the timer reset in Step S161 or the initiation of the latest-biological-data display process. It is to be understood that the above time values, such as 3 seconds and 30 seconds, may be appropriately set in consideration of user-friendliness.

When the determination in Step S163 is YES or either one of the up key 61 and the down key 62 is pressed down, the process advances to Step S167. In Step S167, the timer initially reset in Step S161 is reset again. Thus, the timer determines a lapsed time from the pressing of the up key 61 or the down key 62.

Then, in Step S168, it is determined whether the second designation number Y about the illumination mode has a value of “2”. When the determination in Step S168 is YES or the value of the second designation number Y is “2”, the process skips Step S169 and advances to Step S170. If the determination in Step S168 is NO or the value of the second designation number Y is not “2“(Y=“1”), the process will advance to Step S169. As described above, the initial value of the second designation number Y is set at “1”. Thus, if the process advances to Step S168 after either one of the up key 61 or the down key 62 is pressed down for the first time, the process will obviously advance to Step S169.

In Step S169, the value of the second designation number Y is incremented by one or becomes “2”. Thus, the light emitter 9 is activated in the illumination mode defined by the second designation number Y of “2” (or blinked on and off at a medium speed). After the second designation number Y is changed to “2”, Step S169 will be skipped according to the determination in Step S168.

In Step S170, the first designation number X is incremented by one. Specifically, when the first designation number X has a current value of “1”, the value is changed to “2”. When the first designation number X has a current value of “2”, the value is changed to “3”. As described above, the first designation number X is programmed in such a manner as to be retuned to “1” when it reaches “9”. After completion of Step S170, the process advances to Step S166. In Step S166, it is determined whether 30 seconds have passed from the timer reset in Step S167, or whether 30 seconds have passed from the pressing of the up key 61 or the down key 62.

In the process flow between Step S163 and Step S166 through Step S167, the display control of the biological data and the illumination control of the light emitter 9 according to the user's selection operation are performed. That is, one of the designation numbers X (or one of the nine types of biological data) is selected according to the user's selection operation or the operation of pressing down the up key 61 or the down key 62, and the biological data freely selected by the user will be displayed on the liquid-crystal display screen 5 until the user performs a next selection operation. Thus, the biological data to be displayed on the liquid-crystal display screen 5 is switched every time the user presses down the up key 61 or the down key 62. Simultaneously, the light emitter 9 assigned with the same first designation number X as that of the selected biological data is activated in the illumination mode defined by the second designation number of “2” (or blinked at a medium speed). These display and illumination controls will continue for total 30 seconds from the timer reset in Step S167 or the time when the user lastly presses down the up key 61 or the down key 62. For example, the program for switching the biological data may be configured to increment the first designation number X one-by-one when the up key 61 is pressed down, and decrement the first designation number X one-by-one when the down key 62 is pressed down. This program makes it possible to reverse the order of biological data switching depending on the pressed keys.

When the determination in Step S166 is YES or 30 seconds have passed, the latest-biological-data display process is completed and thereby the entire biological-data measurement process is completed.

Returning to the flowchart of FIG. 4, a process to be performed when the determination in Step S13 is YES or the set key 63 is pressed. In this process, the set key 63 is pressed to display past biological data stored in the storage device 16. When the determination in Step S13 is YES or the set key 63 is pressed, the process advances to Step S17.

In Step S17, a measurement date of biological data to be displayed is input. Specifically, numerals representing year, month and day are displayed on the liquid-crystal display screen 5, and the user increases/reduces each of the numerals using the up key 61 and/or the down key 62 to adjust the numerals to the past measurement date, and sets the adjusted measurement date using the set key 63. It is to be understood that the apparatus 1 may be designed to input the member of lapsed days from this measurement or the number of times of measurements before this measurement may be input, in place of the measurement date.

Then, in Step S18, the biological data having the measurement date input in Step S17 is read out. If no corresponding data is stored, the process may be terminated after displaying an error message indicative of such information, or may return to Step S17.

Then, in Step S19, the past biological data read in Step S18 are displayed on the liquid-crystal display screen 5, and simultaneously the corresponding light emitters 9 are activated. A control in Step S19 will be described below with reference to the flowchart of FIG. 6. The flowchart of FIG. 6 is fundamentally similar to that of FIG. 5, and its description about a different point will be simply described.

In Step S191, the timer is reset in the same manner as that in Step S161.

Then, in Step S192, the display control of each of the biological data and the illumination control of the corresponding light emitter 9 are performed according to the first designation number X in the same manner as that in Step S162, except that the biological data to be displayed are the past biological data read in Step S18, and the illumination mode (lighting pattern) of the light emitters 9 is determined by a third designation number Z instead of the second designation number Y The third designation number Z is programmed in such a manner as to be initially set to “1” and incremented by one in the after-mentioned Step S199, wherein the light emitters are blinked at a medium speed (activated in a medium-speed blinking mode) when the third designation number Z is “1”, and blinked at a high speed (activated in a high-speed blinking mode) when the third designation number Z is “2”. In this manner, two different illumination modes are prepared for the latest-biological-data display and the past-biological-data display, and the two different third designation numbers Z are provided to set the different illumination modes, respectively. Thus, the illumination mode of the light emitters 9 during the latest-biological-data display can be changed from that during the past-biological-data display.

Then, in Step S193, it is determined whether the up key 61 or the down key 62 is pressed down, in the same manner as that in Step S163. If the determination in Step S193 is NO or none of the up key 61 and the down key 62 is pressed down, the process will advance to Step S194. When the determination in Step S193 is YES or either one of the up key 61 and the down key 62 is pressed down, the process advances to Step S197.

In Step S194, it is determined whether the third designation number Z has a value of “2”. When the determination in Step S194 is NO or the third designation number Y≠“2”, the process advances to Step S195. If the determination in Step S194 is YES or the third designation number Y=“2”, the process will skip Step S195 and advance to Step S196.

In Step S195, it is determined whether 3×x seconds have passed from the timer reset in Step S191, in the same manner as that in Step S165. When the determination in Step S195 is YES or 3×x seconds have passed, the process advances to Step S196 through Step S200. If the determination in Step S195 is NO or 3×x seconds have not passed, the process will advance to Step S196 without going through Step S200.

In Step S196, it is determined whether 30 seconds have passed the timer reset in Step S191 or the after-mentioned Step S197, in the same manner as that in Step S166. If the determination in Step S196 is YES or 30 seconds have passed, the past-biological-data display process will be completed. When the determination in Step S196 is NO or 30 seconds have not passed, the process returns to Step S192.

In Step S197, the timer is reset in the same manner as that in Step S167, and then the process advances to Step S198.

Then, in Step S198, it is determined whether the third designation number Z has a value of “2”. If the determination in Step S198 is NO or the value of the second designation number Y≠“2”, the process will advance to Step S199. When the determination in Step S198 is YES or the value of the second designation number Y=“2”, the process skips Step S199 and advances to Step S200.

In Step S199, the value of the third designation number Z is incremented by one or becomes “2”, in the same manner as that in Step S170, and then the process advances to Step S196.

When the determination in Step S196 is YES or 30 seconds have passed, the past-biological-data display process is completed and thereby the entire biological-data measurement process is completed.

FIGS. 7(A) to 7(D) illustrates the display of the liquid-crystal display screen 5 and the illumination mode of the light emitter 9 in the apparatus 1. The light emitters 9 or the nine LEDs 91 to 99 are disposed adjacent to the liquid-crystal display screen 5. The LEDs 91 to 99 are covered, respectively, with nine light-transparent films 91a, 92a, 93a, 94a, 95a, 96a, 97a, 98a, 99a. Each of the films 91a to 99a has a mark representing the biological data associated with the LED covered therewith.

FIG. 7(A) shows one example of a display about body weight (first designation number X=” 1”) as one of the biological data. A value “73.1” indicative of body weight and its unit “kg” are displayed on the liquid-crystal display screen 5, and the LED 91 covered with the film 91a having a mark representing body weight (weight mark) is lighted or activated in a normal illumination mode (second designation number Y=1). That is, this screen currently displays a body weight data which is a latest data just after being measured, and will be automatically switched to display another biological data (first designation number X=“2”) after 3 seconds.

FIG. 7(B) shows one example of a display about body fat percentage (first designation number X=“2”) as another of the biological data. A value “21.7” indicative of body fat percentage and its unit “%” are displayed on the liquid-crystal display screen 5, and the LED 92 covered with the film 92a having a mark representing body fat percentage (mark having a white symbol “%” on a human-body-shaped colored background) is activated in the medium-speed blinking mode (second designation number Y=2). That is, this screen currently displays a body fat percentage data which is a latest data just after being measured, and will be continuously displayed for 30 seconds unless the user presses either one of the up key 61 or the down key 62.

FIG. 7(C) shows one example of a display about bone mass (first designation number X=“8”) as still another of the biological data. A value “4.7” indicative of bone mass and its unit “kg” are displayed on the liquid-crystal display screen 5, and the LED 98 covered with the film 98a having a mark representing bone mass (mark having while bone-shaped lines on a human-body-shaped colored background) is activated in the low-speed blinking mode (third designation number Z=“1”). That is, this screen currently displays a bone mass data which is stored in the storage device 16 after being measured at a certain past time, and will be automatically switched to display another biological data (first designation number X=“9”) after at least 3 seconds.

FIG. 7(D) shows one example of a display about visceral fat level (first designation number X=“9”) as yet another of the biological data. A value “9” indicative of visceral fat level is displayed on the liquid-crystal display screen 5, and the LED 99 covered with the film 99a having a mark representing visceral fat level (mark having a white abdominal section on a human-body-shaped colored background) is activated in the high-speed blinking mode (third designation number Z=2). That is, this screen currently displays a visceral fat level data which is stored in the storage device 16 after being measured at a certain past time, and will be continuously displayed for 30 seconds unless the user presses either one of the up key 61 or the down key 62.

As described above, in this apparatus 1, data measurement means primarily composed of the input device 6, the weight sensor 13, the current-supply electrodes 31, 32, the current-supply circuit 11, the measurement electrodes 41, 42, the voltage measurement circuit 12, and the control circuit 10, measures the plural (nine) types of biological data of a user, as in Steps S4 to S6 and Steps S14 and S15. Then, data selection means primarily composed of the input device 6 and the control device 10 selects one of the measured biological data as in Steps S163 to S170 or Steps S193 to S200, and data display means primarily composed of the liquid-crystal display screen 5 and the control device 10 displays the selected biological data as in Step S162 or S192. In particular, the plurality (nine) of light emitters 9 primarily composed of the LEDs 91 to 99 are provided correspondingly to the biological data, and illumination control means primarily composed of the control device 10 activates the light emitter 9 corresponding to the selected biological data, as in Step S162 or S192.

The LEDs 91 to 99 constituting the light emitters 9 are covered, respectively, with the light-transparent films 91a to 99a each having a mark representing the corresponding biological data.

Further, the data selection means primarily composed of the input device 6 and the control device 10 includes automatic data-selection means adapted to automatically select each of the plural types of biological data in turn at given time (3-second) intervals, as in Steps S165 and S170 or Steps S195 and S200, and optional data-selection means adapted to arbitrarily select one of the plural types of biological data according to a selection operation (pressing of the up key 61 or the down key 62) performed by the user, as in Steps S163 and S170 or Steps S193 and S200. The illumination control means primarily composed of the control device 10 activates the light emitters 9 in a first illumination mode (normal illumination mode or low-speed blinking mode) when the selection of the biological data is performed by the automatic data-selection means, and in a second illumination mode (medium-speed blinking mode or high-speed blinking mode) when the selection of the biological data is performed by the optional data-selection means, as in Steps S162 and S169 or Steps S192 and S199.

Furthermore, data storage means primarily composed of the storage device 16 stores the plural types of biological data measured, as in Step 15, and the data selection means primarily composed of the input device 6 and the control device 10 includes latest-data selection means adapted to select one of the latest biological data measured by the data measurement means, as in Steps S13 and S16, and past-data selection means adapted to select one of past biological data stored on the data storage means. The illumination control means activates the light emitters 9 in a third illumination mode (normal illumination mode and medium-speed blinking mode) when the selection of the biological data is performed by the latest-data selection means, and in a fourth illumination mode (low-speed blinking mode or high-speed blinking mode) when the selection of the biological data is performed by the past-data selection means, as in Steps S162 and S192.

While the living body measuring apparatus of the present invention has been described in connection with one specific embodiment, the present invention is not limited to the specific embodiment, but various modifications and changes may be made therein without departing from the spirit and scope thereof as set forth in appended claims. For example, the number of the biological data types is not limited to nine, but may be less than or greater than nine. The light emitter is not limited to an LED, but any other suitable type, such as a light bulb, may be used. Further, the mark in the light-transparent film covering each of the light emitters may be a character representing the associated biological data. The change in illumination mode of the light emitters is not limited to switching between normal lighting and blinking, but may be any other suitable means, for example switching between different colors. Furthermore, the present invention may be applied to a living body measuring apparatus having a plurality of dedicated selection keys corresponding, respectively, to plural types of biological data. In this case, the dedicated selection keys and the light emitters may be integrated together. It is also understood that the present invention may be widely applied to not only the so-called body composition analyzer utilizing bioelectric impedance but also any apparatus for measuring plural types of biological data (e.g. a pedometer designed to measure the number of steps, consumed energy and required energy).

What is claimed is:

1. A living body measuring apparatus comprising:
data measurement means for measuring plural types of biological data of a user;
data selection means for selecting one of said plural types of measured biological data;
data display means for displaying said selected biological data, wherein said apparatus further comprises:
a plurality of light emitters corresponding to said plural types of biological data; and
illumination control means for activating the light emitters corresponding to said biological data selected by said data selection means.

2. The living body measuring apparatus according to claim 1, wherein each of said plurality of light emitters is covered by a transparent cover having characters or a sign representing biological data corresponding to said light emitter.

3. The living body measuring apparatus according to claim 1 or 2, wherein said data selection means comprises:
automatic data-selection means for automatically selecting each of the plural types of biological data in turn at given time intervals; and
optional data-selection means for selecting one of the plural types of biological data according to a selection operation performed by the user, and wherein said illumination control means is operable to change a illumination mode of said light emitters depending on whether the selection of the biological data is performed by said automatic data-selection means or said optional data-selection means.

4. The living body measuring apparatus according to claim 1 or 2, further comprising data storage means for storing the plural types of biological data measured by said data measurement means, wherein said data selection means comprises:
latest-data selection means for selecting one of the latest biological data measured by said data measurement means; and
past-data selection means for selecting one of past biological data stored on said data storage means,
and wherein said illumination control means is operable to change a illumination mode of said light emitters depending on whether the selection of the biological data is performed by said latest-data selection means or said past-data selection means.

5. The living body measuring apparatus according to any of claims 1 to 4, wherein said data measurement means comprises:
an input key for inputting at least a body height data of the user;
a weight sensor for measuring a body weight data of the user; and
a combination of an electrode and an electric circuit, for measuring a data about an impedance between two legs of the user,
wherein said plural types of biological data are a plurality of index values about a body composition of the user calculated based on at least said body height, body weight and impedance data.
